(19) United States
(12) Patent Application Publication
(10) Pub. No.: US 2018/0160885 A1
(43) Pub. Date: Jun. 14, 2018

(54) ENDOSCOPY BASED MEDICAL DEVICES, METHODS, AND INNOVATIONS

(71) Applicant: Prosper Abitbol, Boca Raton, FL (US)

(72) Inventor: Prosper Abitbol, Boca Raton, FL (US)

(21) Appl. No.: 15/841,264

(22) Filed: Dec. 13, 2017

Related U.S. Application Data
(60) Provisional application No. 62/433,321, filed on Dec. 13, 2016.
(60) Provisional application No. 62/434,397, filed on Dec. 14, 2016.

(51) Int. Cl. A61B 1/00; A61B 50/13; A61B 1/05; A61M 25/00; A61B 1/06; A61B 10/04; A61B 1/018

(52) U.S. Cl. CPC A61B 1/00131; A61B 50/13; A61B 1/05; A61B 1/00045; A61B 1/0011; A61B 1/00133; A61B 2090/3618; A61B 1/00009; A61B 1/0669; A61B 1/00147; A61B 10/04; A61B 1/018; A61M 2025/0046; A61M 25/0045

(57) ABSTRACT

Disclosed are a system, a device, and a method of a lubricant applicator device to be used with an insertion tube of an endoscope. In one embodiment, a video system center having a processor and a memory; a monitor coupled with the video system center; a light source coupled with the video system center; an endoscope coupled the video system center having a control section; an insertion tube having a camera at a distal end of the insertion tube that permits viewing of an area of an internal structure that is directly in front of the camera; and a mirror to be mechanically extendable at a distance from the camera and reflected backward, so that the camera permits viewing of an area behind the camera through a reflection of an image produced by the mirror when an operator of the endoscope utilizes the control section of the endoscope.

### CLAIM OF PRIORITY

This patent application hereby incorporates by reference the entirety of the disclosures of the following cases and each of the cases on which they depend and further claims priority, incorporate by reference, and claims priority from:
a. U.S. Provisional Patent Application No. 62/430,932 titled “LUBRICANT APPLICATOR FOR ENDOSCOPE” filed on Dec. 7, 2016;
b. U.S. Provisional Patent Application No. 62/433,321 titled “VIEWING AID FOR ENDOSCOPE, AND METHOD” filed on Dec. 13, 2016; and c. U.S. Provisional Patent Application No. 62/434,397 titled “AUTOMATING ENDOSCOPY INSTRUMENT INSERTION AND EXTRACTION THROUGH THE INSTRUMENT CHANNEL OF AN ENDOSCOPE” filed on Dec. 14, 2016.

### FIELD OF TECHNOLOGY

The disclosure relates broadly to medical devices and, more particularly to a system, a device, and a method of endoscopy based medical devices and innovations.

### BACKGROUND

An endoscopy may be a procedure in which a body is examined internally using a medical device called an endoscope. An insertion tube of the endoscope may be a long, flexible tube which is inserted into a patient during the endoscopy. Before and during insertion, lubricant (e.g., a gel material) may be applied to the insertion tube. A doctor, nurse, and/or technician may have to manually apply the lubricant onto the insertion tube by placing the lubricant directly on the tube before each use of the endoscope. This may be a time consuming and repetitive process.

In addition, applying the lubricant may be a messy process (e.g., excess lubricant may drip onto a floor). The user of the endoscope may need to change gloves during each and every use of the insertion tube. The user's hands and clothing might get sticky and covered with lubricant. Another disadvantage of applying the lubricant multiple times during use of the endoscope may be an increased risk of contamination and a prolonged set up time. Further, during insertion of the endoscope and during subsequent procedures, it may also be important to grasp and manipulate a portion of the insertion tube which is external to the patient. This can be problematic if the portion of the insertion tube external to the patient is difficult to turn or is slippery.

The endoscope may be an illuminated optical, typically slender and tubular instrument used to look deep into the body and used in procedures called an endoscopy. The endoscope may be guided through (into) a body cavity via mechanisms internal to the tubular instrument that are controlled externally by the user.

One type of endoscope is the colonoscope. A colonoscope may comprise a CCD camera or a fiber optic camera on a flexible tube passed through the anus. It can provide a visual diagnosis (e.g. ulceration, polyps) and grants the opportunity for biopsy or removal of suspected colorectal cancer lesions. Colonoscopy can remove polyps as small as one millimeter or less.

As used herein, the term “area” refers to an area (e.g., colon) of a patient being examined and/or treated. Generally, the area will be examined looking, for example, for polyps. Snare-type devices may be extended out of the end of the endoscope to lasso the polyps, and a heated wire may be used to cauterize the polyps. As may be used herein, the term “scope” refers to an endoscope.

### SUMMARY

Disclosed are a system, a device, and a method of endoscopy based medical devices and innovations. In one aspect, a video system center having a processor and a memory; a monitor coupled with the video system center; a light source coupled with the video system center; an endoscope coupled the video system center having a control section; an insertion tube having a camera at a distal end of the insertion tube that permits viewing of an area of an internal structure that is directly in front of the camera; and a mirror to be mechanically extendable at a distance from the camera and reflected backward, so that the camera permits viewing of an area behind the camera through a reflection of an image produced by the mirror when an operator of the endoscope utilizes the control section of the endoscope.

In another aspect, a lubricant applicator system includes a tubular member, an enlarged bulbous region, and a handle region. The tubular member surrounds a portion of the endoscope insertion tube (e.g., the endoscope insertion tube having a mirror that can be mechanically extended in front of a camera so that the camera extended a distance from the camera and reflected backward, so that the camera can view an area behind the camera through a reflection of an image produced by the mirror when an operator of the endoscope utilizes a control section of an endoscope). The enlarged bulbous region is adjacent to a distal end of the tubular member. The handle region is adjacent to a proximal end of the tubular member. The tubular member is such that the tubular member is extendable (e.g., flexibly extendable, manipulatable, etc.) over the portion of the endoscope insertion tube to snugly surround the portion of the endoscope insertion tube. The enlarged bulbous region is pre-filled with a gel material that is releasable onto a surface area of the endoscope insertion tube encompassed by the enlarged bulbous region. The handle region adjacent to the proximal end of the tubular member extends from the enlarged bulbous region provides a non-slip exterior surface to enable a human hand of an operator to grasp the handle region and manipulate the endo scope insertion tube while rotatably affixing a thumb of the hand onto the enlarged bulbous region to release the gel material when a pressure is applied to the enlarged bulbous region using the thumb.

A flow rate of the gel material onto the surface area of the endoscope insertion tube encompassed by the enlarged bulbous region may be proportional to the pressure applied to the enlarged bulbous region by the thumb of the human hand of the operator. The lubricant applicator device may be any one of a single use lubricant applicator device and/or a multiple use lubricant applicator device.

The gel material may be refillable in the enlarged bulbous region when the lubricant applicator device is the multiple use lubricant applicator device. The gel material may be pre-filled in the enlarged bulbous region at a factory when the lubricant applicator device is the single use lubricant applicator device.

The tubular member may be a single mold assembly including both the enlarged bulbous region and the handle region. The tubular member may be formed of a material including a rubber, a polymeric flexible material, a nylon material, a biodegradable material, a silicon based material, and/or a semi-rigid material. The insertion tube may be inserted into the lubricant applicator device axially through the proximal end of the tubular member and continuing through the tubular member. The insertion tube may emerge from the distal end of the tubular member adjacent to the enlarged bulbous region.

A section of the insertion tube encapsulated by the handle region may remain segregated from the gel material of the enlarged bulbous region such that an exterior surface of the handle region will remain unaffected by an extraction of the gel material from the enlarged bulbous region when the pressure of the thumb of the operator is applied on the enlarged bulbous region. The pressure of the thumb of the operator may cause the gel material to release onto the surface area of the endoscope insertion tube encompassed by the enlarged bulbous region of the lubricant applicator device.

The endoscope insertion tube may be transgressible through the lubricant applicator device when it is pushed through the tubular member from the proximal end of the tubular member. The gel material may be a water-soluble gel that is gentle on mucosal surfaces of a patient. The enlarged bulbous region may be a chamber that includes the gel material.

In yet another aspect, a lubricant applicator device includes a tubular member, an enlarged bulbous region and a handle region. The enlarged bulbous region is at a distal end of the lubricant applicator device. The handle region is at a proximal end of the lubricant applicator device. The tubular member surrounding a portion of an endoscope insertion tube is such that the tubular member is extendable (e.g., flexibly movable, manipulatable, stretchable, placeable, etc.) over the portion of the endoscope insertion tube to surround the portion of the endoscope insertion tube. The handle region provides a non-slip exterior surface to enable a human hand of an operator to grasp the handle region and manipulate the endoscope insertion tube while rotatably affixing a thumb of the hand onto the enlarged bulbous region to release the gel material when a pressure is applied to the enlarged bulbous region using the thumb.

In a further aspect, a method of forming a lubricant applicator device includes generating a single mold having a tubular member, an enlarged bulbous region, and a handle region. The enlarged bulbous region is at a distal end of the lubricant applicator device. The handle region is at a proximal end of the lubricant applicator device. The tubular member surrounding a portion of an endoscope insertion tube is such that the tubular member is placed over the portion of the endoscope insertion tube surrounding the portion of the endoscope insertion tube. The handle region provides a non-slip exterior surface to enable a human hand of an operator to grasp the handle region and manipulate the endoscope insertion tube while rotatably affixing a thumb of the hand onto the enlarged bulbous region to release the gel material when a pressure is applied to the enlarged bulbous region using the thumb.

The lubricant applicator device may have a sideways water-tower like shape. The lubricant applicator device may be formed of a material including a rubber, a polymeric flexible material, a nylon material, a biodegradable material, a silicon based material, and/or a semi-rigid material.

The methods, devices, and systems disclosed herein may be implemented in any means for achieving various aspects, and may be executed in a form of a machine-readable medium embodying a set of instructions that, when executed by a machine, cause the machine to perform any of the operations disclosed herein. Other features will be apparent from the accompanying drawings and from the detailed description that follows.

### BRIEF DESCRIPTION OF THE DRAWINGS

The embodiments of this invention are illustrated by way of example and not limitation in the figures of the accompanying drawings, in which like references indicate similar elements and in which:

FIG. 1 is an apparatus view of a lubricant applicator device, according to an embodiment.

FIG. 2 is an endoscope view illustrating the environment of using the lubricant applicator device of FIG. 1 with endoscope, according to an embodiment.

FIG. 3 is a system view illustrating the environment of using the lubricant applicator device of FIG. 1 with endoscope while operating, according to an embodiment

FIG. 4 is an operation view explaining the working of the lubricant applicator device of FIG. 1, according to an embodiment.

FIG. 5 is a cross-sectional view of the lubricant applicator device of FIG. 1, according to an embodiment.

FIG. 6 is a gland view illustrating the gel chamber/gland at the inside of the lubricant applicator device of FIG. 1 and the chamber retaining wall for containing the gel at the ‘slice’, according to an embodiment.

FIG. 7 is an elevational view of the lubricant applicator device of FIG. 1 illustrating the slots of the lubricant applicator device of Figure, according to an embodiment.

FIG. 8 is an operation view of the lubricant applicator device of FIG. 1 illustrating the opening-up of the lubricant applicator device of FIG. 1, according to an embodiment.

FIG. 9 is a removable corner view of the lubricant applicator device of FIG. 1 illustrating the removal of plastic tube to expose the gel of the lubricant applicator device at the gel corner to the endoscope insertion tube of the endoscope, according to an embodiment.

FIG. 10 is a peeling view of the lubricant applicator device of FIG. 1 illustrating the removal and/or mounting of the lubricant applicator device, according to an embodiment, according to one embodiment.

FIG. 11 is an extrusion view of the lubricant applicator device of FIG. 1 illustrating the extrusion end of the lubricant applicator device, according to an embodiment.

FIG. 12 is a radial view of the lubricant applicator device of FIG. 1, according to one embodiment.

FIG. 13 is a typical view of an endoscope FIG. 2 illustrating the component parts of the endoscope, according to an embodiment.

FIG. 14 is an end view of the nozzle of the endoscope of FIG. 2 illustrating the component parts of the endoscope, according to an embodiment.

FIG. 15 illustrates a snare device connected to the endoscope of FIG. 2, according to an embodiment.

FIG. 16 illustrates a mirror element of the snare device connected to the endoscope of FIG. 2, according to an embodiment.

FIG. 17 is a cross-sectional view of the mirror element of the snare device of FIG. 16, according to an embodiment.

FIG. 18 illustrates the contraction view of the snare device of FIG. 16, according to an embodiment.

FIG. 19 illustrates a biopsy device mechanism connected to the endoscope of FIG. 2, according to an embodiment.

FIG. 20 illustrates an operation view of an elongated biopsy device connected to the endoscope of FIG. 2, according to an embodiment.

Other features of the present embodiments will be apparent from the accompanying drawings and from the detailed description that follows.

### DETAILED DESCRIPTION

Disclosed are a device, and/or a method of a removability and transportability of a lubricant applicator device 150 of an endoscope 250. In one embodiment, a video cart system 350 includes a video system center 314 having a processor and a memory, a monitor 318 coupled with the video system center 314, a light source 316 coupled with the video system center 314, an endoscope 250 coupled the video system center 314 having a control section 306, an insertion tube 110 having a camera at a distal end 106 of the insertion tube 110 that can view an area of an internal structure that is directly in front of the camera, and a mirror that can be mechanically extended a distance from the camera and reflected backward, so that the camera can view an area behind the camera through a reflection of an image produced by the mirror 1602 when an operator 320 of the endoscope 250 utilizes the control section 306 of the endoscope 250.

In another embodiment, the lubricant applicator device 150 includes a tubular member 100, an enlarged bulbous region 102, and a handle region 104. The tubular member 100 surrounds a portion of an endoscope insertion tube 110 having a mirror element 1602 that can be mechanically extended a distance from the camera and reflected backward, (e.g., near the nozzle 202) so that the camera can view an area behind the camera through a reflection of an image produced by the mirror when an operator of the endoscope 250 utilizes a control section 306 of an endoscope 250. The tubular member 100 is extendable (e.g., flexibly, extendable, manipulatable, etc.) over the portion of the endoscope insertion tube 110 to snugly surround the portion of the endoscope insertion tube 110. The enlarged bulbous region 102 is adjacent to a distal end 106 of the tubular member 100. The enlarged bulbous region 102 is pre-filled with a gel material 112 that is releasable onto a surface area of the endo scope insertion tube 110 encompassed by the enlarged bulbous region 102. The handle region 104 adjacent to a proximal end 108 of the tubular member 100 extends from the enlarged bulbous region 102. The handle region 104 provides a non-slip exterior surface 114 to enable a human hand 400 of an operator 320 to grasp the handle region 104 and manipulate the endoscope insertion tube 110. A thumb 402 of the hand (e.g., human hand 400) of the operator 320 onto the enlarged bulbous region 102 releases the gel material 112 when a pressure is applied to the enlarged bulbous region 102 using the thumb 402. A plastic tube 702 is provided in a cavity of the enlarged bulbous region 102 closest to a location of the endoscope insertion tube 110. The plastic tube 702 seals the gel material 112 prior to usage in a chamber (e.g., gel chamber/gland 604) of the enlarged bulbous region 102.

The tubular member 100 may have a set of topological features. The set of topological features of the tubular member 100 may include bumps and/or grooves 502 to deliver the gel (e.g., gel material 112) onto the endoscope insertion tube 110 when the pressure is applied to the enlarged bulbous region 102 using the thumb 402. A flow rate of the gel material 112 onto the surface area of the endoscope insertion tube 110 encompassed by the enlarged bulbous region 102 may be proportional to the pressure applied to the enlarged bulbous region 102 by the thumb 402 of the operator 320.

The proximal end 108 and/or the distal end 106 of the lubricant applicator device 150 may be flared to facilitate insertion of the endoscope insertion tube 110 into the tubular member 100. The plastic tube 702 may be optionally slit (e.g., slot 704) to permit a lateral removal of the plastic tube 702 from the endoscope insertion tube 110. The plastic tube 702 may serve as a guide when the lubricant applicator device 150 is applied on the endoscope insertion tube 110. The gel material 112 may be pre-filled in the enlarged bulbous region 102 at a factory.

The tubular member 100 may be separated along its length through a slit (e.g., slot 704) formed in a body of the tubular member 100. The tubular member 100 may facilitate lateral installation of the lubricant applicator device 150 on the endoscope insertion tube 110. The slit (e.g., slot 704) may permit the lateral removal of the lubricant applicator device 150 from the endoscope insertion tube 110. The lubricant applicator device 150 may be peelable (e.g., device can be removed off (or mounted onto) the scope by ‘peeling’ 1102) sideways off the endoscope insertion tube 110 after use. The tubular member 100 may be a single mold assembly including of both the enlarged bulbous region 102 and the handle region 104. The tubular member 100 may be formed of a material including a rubber, a polymeric flexible material, a nylon material, a biodegradable material, a silicon based material, and/or a semi-rigid material.

The endoscope insertion tube 110 may be inserted into the lubricant applicator device 150 axially through the proximal end 108 of the tubular member 100 and continuing through the tubular member 100. The endoscope insertion tube 110 may emerge from the distal end 106 of the tubular member 100 adjacent to the enlarged bulbous region 102. A section of the endoscope insertion tube 110 encapsulated by the handle region 104 may remain segregated from the gel material 112 of the enlarged bulbous region 102 such that an exterior surface (e.g., non-slip exterior surface 114) of the handle region 104 remains unaffected by an extraction of the gel material 112 from the enlarged bulbous region 102. The pressure of the thumb 402 of the operator 320 may cause the gel material 112 to release onto the surface area of the endoscope insertion tube 110 encompassed by the enlarged bulbous region 102 of the lubricant applicator device 150.

The endoscope insertion tube 110 may be transgressible through the lubricant applicator device 150 when it is pushed through the tubular member 100 from the proximal end 108 of the tubular member 100. The non-slip exterior surface 114 of the handle region 104 may include a ridge pattern and/or a dappled pattern to improve grip during manipulation of the endoscope insertion tube 110 by the operator 320. The enlarged bulbous region 102 may be a chamber (e.g., gel chamber/gland 604) that comprises the gel material 112. The gel material 112 may be a water-soluble gel that is gentle on mucosal surfaces of a patient.

In yet another embodiment, a lubricant applicator device 150 includes a tubular member 100, a handle region 104, and a non-slip exterior surface 114. The tubular member 100 has an enlarged bulbous region 102 at a distal end 106 of the lubricant applicator device 150 and a handle region 104 at a proximal end 108 of the lubricant applicator device 150. The tubular member 100 surrounds a portion of an endoscope insertion tube 110. The tubular member 100 is flexible (e.g., flexibly movable, manipulatable, stretchable, placeable, etc.) over the portion of the endoscope insertion tube 110 to surround the portion of the endoscope insertion tube 110. The handle region 104 provides the non-slip exterior surface 114. The non-slip exterior surface 114 of the handle region 104 enables a human hand 400 of an operator 320 to grasp the handle region 104 and manipulate the endoscope insertion tube 110. A thumb 402 of the hand (e.g., human hand 400) of the operator 320 onto the enlarged bulbous region 102 releases a gel material 112 when a protective shield in a form of a plastic tube 702 is removed and a pressure is applied to the enlarged bulbous region 102 using the thumb 402. The tubular member 100 is formed with a set of topological features. The set of topological features includes bumps and/or grooves 502 to deliver the gel (e.g., gel material 112) onto the endoscope insertion tube 110 when the pressure is applied to the enlarged bulbous region 102 using the thumb 402. The non-slip exterior surface 114 of the handle region 104 includes a ridge pattern and/or a dappled pattern to improve grip during manipulation of the endoscope insertion tube 110 by the operator 320. A plastic shield is provided in a cavity of the enlarged bulbous region 102 to assist in placement of the endoscope insertion tube 110 into the tubular member 100. The plastic shield restricts the gel material 112 in a chamber (e.g., gel chamber/gland 604) of the enlarged bulbous region 102 prior to usage.

In a further embodiment, a method of forming a lubricant applicator device 150 includes generating a single mold having a tubular member 100 with an enlarged bulbous region 102 at a distal end 106 of the lubricant applicator device 150 and a handle region 104 at a proximal end 108 of the lubricant applicator device 150. The tubular member 100 surrounds a portion of an endoscope insertion tube 110. The tubular member 100 is placeable (e.g., flexibly movable, manipulatable, stretchable, etc.) over the portion of the endoscope insertion tube 110 to surround the portion of the endoscope insertion tube 110. The handle region 104 provides a non-slip exterior surface 114 to enable a human hand 400 of an operator 320 to grasp the handle region 104 and manipulate the endoscope insertion tube 110. A thumb 402 of the hand (e.g., human hand 400) onto the enlarged bulbous region 102 releases the gel material 112 when a protective shield in a form of a plastic tube 702 is removed and a pressure is applied to the enlarged bulbous region 102 using the thumb 402. The tubular member 100 is separated along its length through a slit (e.g., slot 704) formed in a body of the tubular member 100. The tubular member 100 facilitates a lateral installation and/or a lateral removal of the lubricant applicator device 150 from the endoscope insertion tube 110.

In the following description, for the purposes of explanation, numerous specific details are set forth in order to provide a thorough understanding of the various embodiments. It will be evident, however to one skilled in the art that the various embodiments may be practiced without these specific details.

An endoscope 250 may be an instrument that can be introduced into the body to give a view of its internal parts. An endoscopy is a procedure where the inside of a body is examined using the endoscope. The endoscope may include an endoscope insertion tube 110 that may have a light source and/or a camera at one end. Images of the inside of the body are relayed to a display screen. The endoscope insertion tube 110 may be guided through (into) a body cavity via mechanisms controlled externally by a user. The endoscope insertion tube 110 can be inserted into the body through a natural opening, such as into the mouth and down the throat, or through the bottom. The endoscope insertion tube 110 can also be inserted through a small cut or incision made in the skin when keyhole surgery is being carried out.

The endoscopy can be used to investigate unusual symptoms, including but not limited to, difficulty swallowing (dysphagia), persistent abdominal pain, chest pain that is not caused by heart-related conditions, persistent nausea and vomiting, unexplained weight loss, vomiting blood, persistent diarrhea, and blood in stool. The endoscope 250 can also help perform certain types of surgery, and/or remove a small sample of tissue for further analysis (e.g., a biopsy).

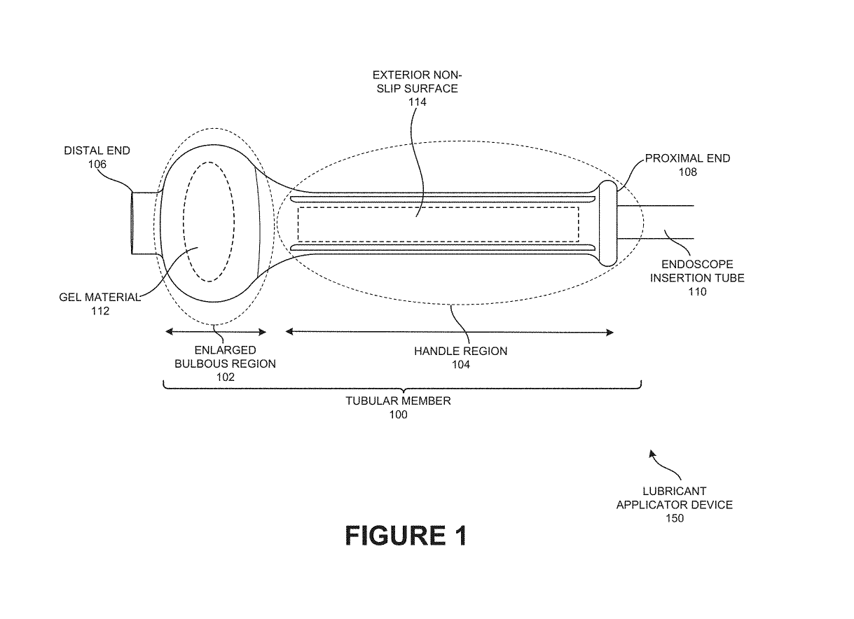

FIG. 1 is an apparatus view of a lubricant applicator device 150, according to an embodiment. Lubricant applicator device 150 illustrating different parts of the lubricant applicator device 150, according to one or more embodiments. Particularly, FIG. 1 illustrates a tubular member 100, an enlarged bulbous region 102, a handle region 104, a distal end 106, a proximal end 108, an endoscope insertion tube 110, a gel material 112, and an exterior non-slip surface 114 of the lubricant applicator device 150, according to one or more embodiments.

Lubricant applicator device 150 may comprise an elongated, generally tubular (or cylindrical) element (or member) 100, having two ends (e.g., proximal end 108, distal end 106). One end (e.g., proximal end 108) may be referred to as the “doctor end” or “back end” or “proximal end” of the tubular element 100. The other end (e.g., distal end 106) may be referred to as the “patient end” or “front end” or “distal end” of the tubular element 100, according to one embodiment.

The tubular element 100 may be made of a flexible rubber (or other elastomer) material, by a molding process. Some suitable materials for the tubular element 100 may include silicon rubber, or other polymeric flexible or semi-rigid material, such as selected from the group consisting of polydimethylsiloxane (PDMS), PMMA (poly(methyl methacrylate)), PS (polystyrene), and PC (polycarbonate). Other materials may include silicon, vitron rubber, and nylon, according to one embodiment. Although particular materials forming the tubular element 100 have been described, embodiments contemplate any number of materials to form the tubular element 100, according to particular needs.

The tubular element 100 be may have a length of approximately four (4) inches (200 mm), and an inner diameter of approximately ⅝ inches (15 mm). The inner diameter of the tubular element 100 may be slightly smaller than the outer diameter of the endoscope insertion tube 110, so that it may securely grip or hug the endoscope insertion tube 110, according to an embodiment.

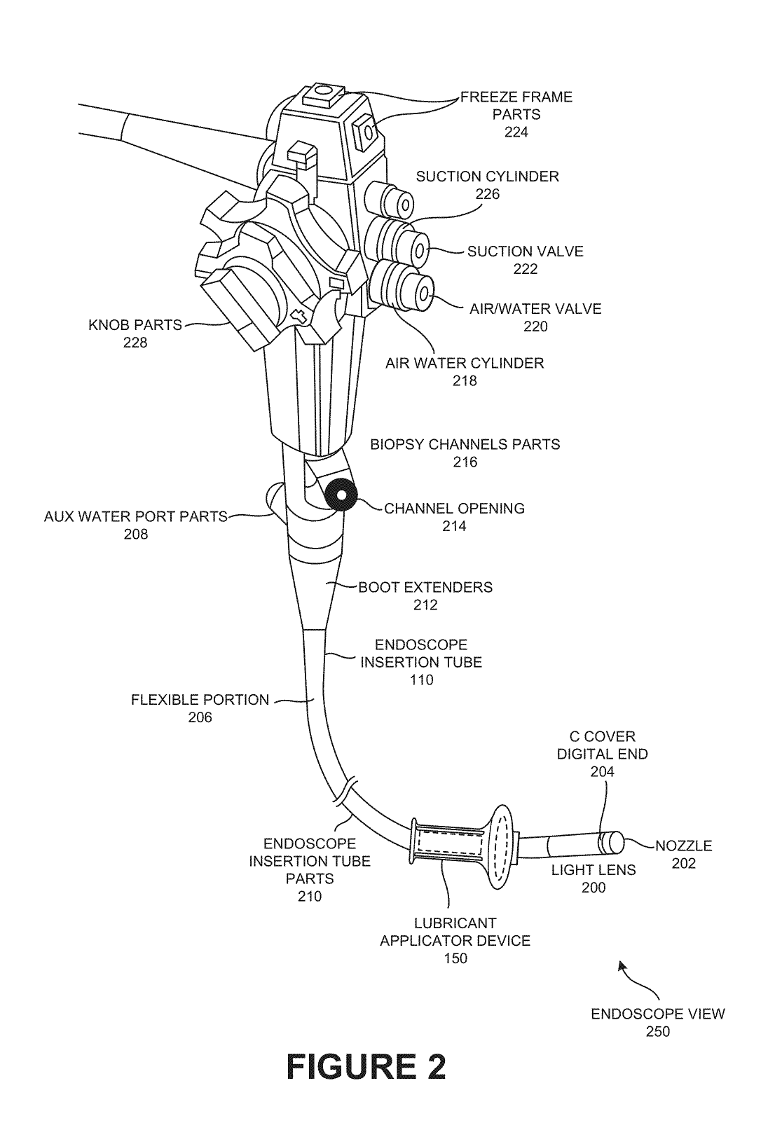

FIG. 2 is an endoscope 250 illustrating a view of the environment of using the lubricant applicator device 150 of FIG. 1 while performing an endoscopy, according to an embodiment. It is noted that the lubricant applicator device 150 must be designed and calibrated to ensure that it does not interfere with a proper operation of the complex operation of the endoscope 250 as described herein. Particularly, FIG. 2 builds on FIG. 1, and further adds a light lens 200 a nozzle 202, a c cover digital end 204, a flexible portion 206, an aux water port 208, an endoscope insertion tube 210, one or more boot extenders 212, a channel opening 214, one or more biopsy channels 216, an air water cylinder 218, air/water valve 220, a suction valve 222, one or more freeze frame parts 224, a suction cylinder 226, and one or more knob parts 228, according to one or more embodiments. The light lens 200 may enable illumination of a target object inside a body. Other items described as 202-228 may be components of the lubricant applicator. Illustrated specifically in FIG. 2 the placement of the lubricant applicator device 150 on a portion of the endoscope insertion tube 210.

The knob parts 228 may be part of the control body of the endoscope 250. This section of the scope may contain the physician controls: angulation control knobs, forceps raiser or elevator lifter, air/water, suction control valves, remote video switches, biopsy port and focus mechanism for fiber optic scopes, tension controller and the control body for holding the endoscope.

The endoscope insertion tube 110 of FIG. 1 as illustrated in FIG. 2 may be along flexible tube inserted into the body during normal use. The length and diameter of the endoscope insertion tube 110 may greatly by model as is dependent upon the anatomy viewed. Similar to the light guide tube, it may be constructed of multiple layers of steel coil, fiber mesh and vulcanized rubber. The thickness of each layer may vary by model and by tube diameter. A clear protective outer layer may be labeled with metric insertion depth marks.

The endoscope insertion tube 110 may be hollow and may provide protection for the delicate internal components including the angulation system, fiber optics, video signal wires and biopsy channel. Sharp bending and over-coiling of the endoscope insertion tube 110 may cause damage to the tube itself, and may damage the internal components. The damage may often indicated by raised ridges or buckles appearing along the tube.

Bending Section—Located at the distal end of the endoscope insertion tube 110, the bending section may be constructed of articulating steel ribs riveted together to form a flexible skeleton-like shell. The shell may be covered with a steel mesh braid and then a thin-walled flexible tube called the bending rubber or sheath. The distal end of the bending section may contain the video chip, optical glass lens systems, fiber optics and/or the termination nozzle of the air/water system. The overall length of the bending section may vary by model but it is usually around four inches long. The outside diameter of most endoscope 250 variants such as gastroscopes, duodenoscopes, and colonoscopes can range from 5.9 to 12.5 mm.

The bending section is the most fragile portion of the endoscope 250 and must be protected from impact. Impact to the distal tip potentially may damage all of the inner components.

The endoscope 250 may have a light transmission component for image illumination. The light may enter the scope, usually at the light guide connector, through the light guide prong and/or paddle connector. The endoscope 250 may have a self-contained battery-powered light source for use when access to a cart system is impractical.

Inside the endoscope 250, light may travel from the light guide prong to the distal end through a light fiber bundle, a collection of thousands of thin, flexible optical glass fibers. Each individual fiber may transport a small portion of light, and together they can produce a very intense bright light. The light may exit the endoscope 250 at the distal end through the light guide lenses, which disperse the light evenly across the visual field. Typical GI scope variants of the endoscope 250 may have two or three light guide bundles.

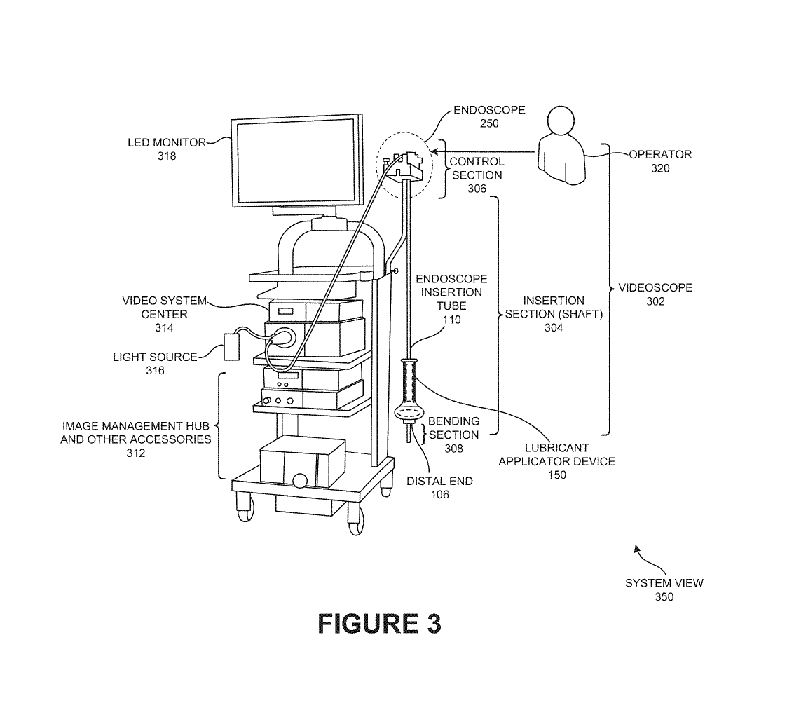

There is no adjustment capacity within the light fiber system in the scope. All light adjustments are made manually on the light source or automatically within the video cart system (e.g., the system view 350 of FIG. 3 may be a video cart system). Damage to the fibers results in reduced light transmission.

The primary function of the endoscope 250 may be imaging. An image may be captured at the distal end (using the objective lens system) and is then transferred to the image transmission device directly behind the lens system. A variant of the endoscope 250 may be a fiber optic endoscope. Fiber optic endoscope variants may use an image fiber bundle (similar to the light fiber bundle) to carry the image to an ocular lens at the control body for viewing. Each of the thousands of fibers may carry a small portion of the image, and a black dot on the image represents one broken fiber.

Video endoscopes (videoscopes) may also be a variant of the endoscope 250. On video endoscope embodiment, the video chip, a charge coupled device (or CCD) is located directly behind the objective lens assembly at the distal end. Such systems may use a CMOS video chip (e.g., complementary metal oxide semiconductor) Here the image may be converted to a digital signal. It may travel through signal wires running through the scope to the light guide connector or video connector. The digital signal then may connect directly to and/or through a cable to the video processor on the cart system. The image can be sent to a variety of monitors, printers or remote locations. The Thinking Cap may provide a visual image of a yellow submarine to illustrate this process. Endoscope video chips may contain from 100,000 to 1 million pixels. More pixels usually may mean better image quality, resolution and color reproduction.

The endoscope 250 may have an angulation system of wires, guided by knobs on the control body. This system may manipulate the bending section for insertion and viewing. The knobs may be connected inside the control body housing to either a pulley-and-wire system or a gear-and-chain system that connects to the angle wires. The angle wires may be braided stainless steel cables that extend from the control body through the endoscope insertion tube 110 and connect to the bending ribs at the distal end. Each wire may control one direction of movement, so two opposite wires work together to provide movement up and down, left and right. The angle wires inside the endoscope insertion tube 110 may be encased within a protective coil pipe that protects the other internal components from the angle wire.

Some colonoscope models of the endoscope 250 may contain an additional wiring component to boost the rigidity of the endoscope insertion tube 110. This rigor may be provided in three levels and is designed to be neutralized before withdrawing the colonoscope after terminal intubation has been achieved. Excess wear and tear on this wired enhancement may be minimized when the stiffness is deactivated for transport, decontamination, reprocessing and storage.

The endoscope 250 may include a biopsy/suction system. The biopsy and suction channels may be part of the same internal system. The biopsy channel may be a straight piece of uniform flexible tubing running from the distal end to the control body. The suction channel may be similar but may run from the control body to the light guide connector. The suction and biopsy channels may be joined in the control body and are controlled by the suction valve controls.

An external suction device connected to the suction channel at the light guide connector may provide the suction pressure needed to perform biopsy, and suctioned material then may travel back through the biopsy channel at the biopsy port opening. The endoscope 250 may include an Air/Water System. The air and water internal channels may be thin flexible tubes running from the light guide connector to the distal end. Although air and water flow through separate tubes, they may be controlled by the same valve on the control body. Air and water exit the scope at the distal end through the air/water nozzles, which may direct the flow over the objective lens.

The endoscope 250 may include a forcep raiser function. The forcep raiser (e.g., elevator) may be at the distal end to deflect accessories passed through the biopsy channel. This small metal arm at the biopsy port opening may be attached to a wire that runs inside the endoscope insertion tube 110, connects to a control lever on the control body, and operates much like the angulation system controls. The forceps raising function may be found on duodenoscopes and endoscopic ultrasound scope variants of the endoscope 250.

Except for the electrical video connections, the entire outer surface of the endoscope 250 may be chemical and fluid resistant. Any seams between parts may be sealed by O-rings, and a colonoscope may contain over forty of these O-rings. The chemical- and fluid-resistant coatings on the flexible tubes may themselves flexible, and some parts of the endoscope 250 may be sealed with epoxy. The ends of the bending rubber may be sealed with silk or nylon braid and then covered with epoxy. Caps or covers may be provided to seal the electrical video connections against fluid. The end result is an endoscope device 250 that, when undamaged, can be immersed completely in water or cleaning/disinfecting fluid. In this way, the instrument can be reprocessed over and over.

According to one embodiment, FIG. 2 is a diagram of an endoscope (e.g., as shown in endoscope view 250). The endoscope 250 may comprise a long flexible endoscope insertion tube 110 having two ends. The endoscope insertion tube 110 may have an outside diameter (OD) of approximately ⅝″ (15 mm). An instrument channel may extend through the extension tube from control apparatus (e.g., control section 306) disposed at a proximal (user, doctor) end 108 of the endoscope insertion tube 110 to the distal (patient) end 106 of the endoscope insertion tube 110, according to one embodiment.

An end cap (e.g., or nozzle 202) may be disposed at the distal end 106 of the endoscope insertion tube 110, and may contain lenses, water jets, etc. (not shown). A distal end 106 portion of the endoscope insertion tube 110 may be flexible, and have an irregular surface. In use, it may be beneficial that the endoscope insertion tube 110 be lubricated (with a gel material 112), without contaminating or clogging the lenses, water jets, etc., according to one embodiment.

FIG. 3 is a system view 350 illustrating the environment of using the lubricant applicator device 150 of FIG. 1 with endoscope while operating according to an embodiment. The lubricant applicator device 150 is connected to the endoscope 250 for the operator 320 to be able to see the inside of human body on an LED monitor 318 while endoscopy, according to an embodiment. Particularly, FIG. 3 builds on FIGS. 1 and 2, and further adds, a videoscope 302, an insertion section (shaft) 304, a control section 306, a bending section 308, the distal end 106, an image management hub and other accessories 312, a video system center 314, a light source 316, an LED monitor 318, and an operator 320, according to one embodiment. The various components 302-318 may be used in a data processing system that controls the endoscope 250 illustrated in FIG. 3. The endoscope 250 in FIG. 3 is illustrated as utilizing the lubricant applicator device 150 of FIG. 1.

While performing an endoscopy, the operator 320 may be able to operate the endoscope 250 to investigate the inside of the human body using control section 306. The lubricant applicator device 150 may be connected to the endoscope 250 through the endoscope insertion tube 110. The endoscope 250 may be connected to an image management hub 312 for a real-time display of endoscopy on the LED monitor 318, according to one embodiment.

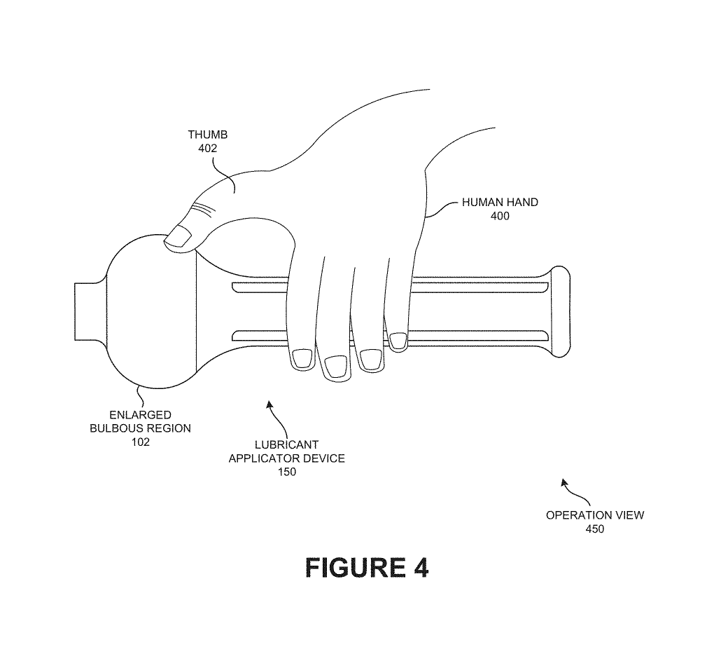

FIG. 4 is an operation view 450 explaining the working of the lubricant applicator device 150 of FIG. 1, according to an embodiment. The enlarged bulbous region 102 of the tubular element 100 may contain gel material 112. The operator 320 may squeeze the enlarged bulbous region 102 of the tubular element 100 by pressing the thumb 402 onto the endoscope insertion tube 110 to dispense the gel material 112, according to one embodiment.

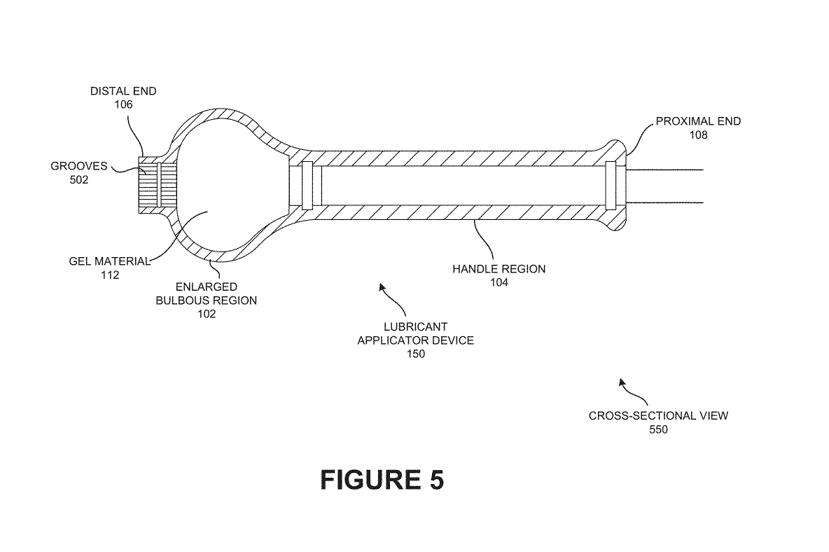

FIG. 5 is a cross-sectional view of the lubricant applicator device 150 of FIG. 1 showing the inside of the lubricant applicator device 150, according to an embodiment.

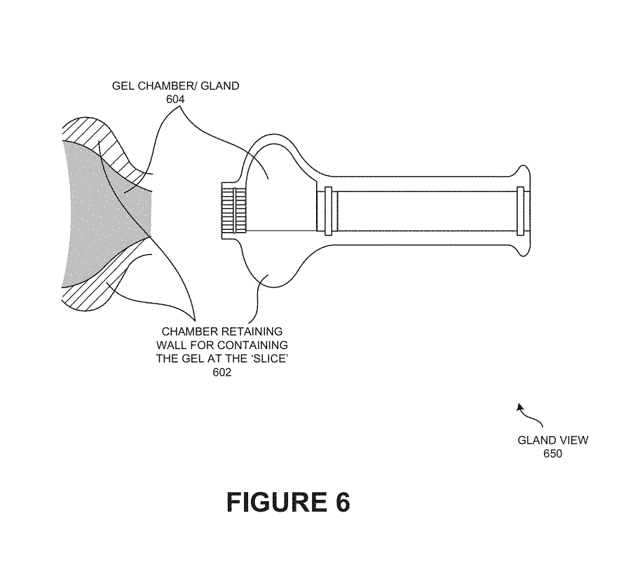

FIG. 6 is a gland view 650 illustrating the gel chamber/gland 604 at the inside of the lubricant applicator device of FIG. 1 and the chamber retaining wall for containing the gel at the ‘slice’ 602, according to an embodiment.

According to an embodiment of FIG. 6, the enlarged bulbous region 102 of the lubricant applicator device 150 at the distal end 106 of the tubular element 100, may be enlarged and have a larger diameter than the remainder of the tubular element 100, to contain the gel material 112 at the chamber retaining wall at the “slice” 602. This portion (as shown in enlarged bulbous region 102) of the tubular element 100 may be referred to as a “gel chamber”, or “gland”, or “chamber”, or “bulb” (as shown in gel chamber/gland 604). When in use, the gel material 112 may be dispensed and/or excreted onto the endoscope insertion tube 110 by squeezing or pressing the gel chamber/gland 604, according to one embodiment.

According to an embodiment of FIG. 6, a bulbous portion (e.g., as shown in enlarged bulbous region 102), such as a portion at the front end (e.g., as shown in distal end 106) of the tubular element 100, may be enlarged (have a larger diameter than the remainder of the tubular element 100), to contain a quantity, such as 75 cc, of lubricant (e.g., gel material 112). This portion (e.g., as shown in enlarged bulbous region 102) of the tubular element 100 may be referred to as a “gel chamber”, or “gland”, or “chamber”, or “bulb” (e.g. as shown in gel chamber/gland 604). In use, gel (e.g., gel material 112) may be dispensed (or excreted) onto the endoscope insertion tube 110 by squeezing (or pressing) the bulb (e.g. as shown in gel chamber/gland 604), according to one embodiment.

The outside surface of the remaining cylindrical (or grip) portion (e.g., as shown in handle region 104) of the tubular element 100 may be provided with ridges, dappled, or the like to improve gripping (e.g., a bicycle hand grip), when the tubular element 100 is installed on the endoscope insertion tube 110. The inside surface of the grip portion (e.g., as shown in handle region 104) may also be provided with ridges, dappled, or the like to improve gripping of the endoscope insertion tube 110. This is advantageous because the endoscope insertion tube 110 has a relatively small diameter (⅝″), making it somewhat difficult to manipulate (particularly if it is lubricated). It may be easier for the user (e.g., operator 320) to manipulate the endoscope insertion tube 110 via the tubular element (or grip) 100 which may have a grip (e.g., non-bulbous portion, handle region 104) diameter of approximately 1¼ inches (30 mm), according to one embodiment.

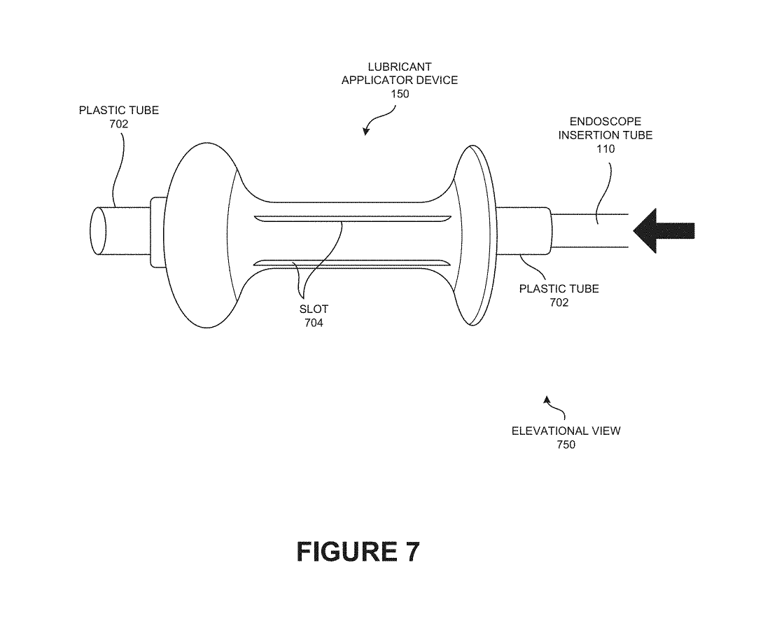

FIG. 7 is an elevational view of the lubricant applicator device of FIG. 1 illustrating the slots of the lubricant applicator device of FIG. 1, according to an embodiment. In one example embodiment, the endoscope 250 may be guided through the plastic tube 702 which is larger in diameter than the endoscope insertion tube 110. The exterior non-slip surface 114 of the handle region 104 of the lubricant applicator device 150 may be provided with ridges, dappled, or the like to improve gripping (e.g., like a bicycle handgrip), when the handle region 104 is installed on the endoscope insertion tube 110. The inside surface of the handle region 104 may also be provided with ridges, dappled, or the like to improve gripping of the endoscope insertion tube 110. This is advantageous because the endoscope insertion tube 110 may have a relatively small diameter (⅝″), making it somewhat difficult to manipulate (particularly if it is lubricated). It may be easier for the operator 320 to manipulate the endoscope insertion tube 110 via the tubular member 100 which may have a grip (non-bulbous portion) diameter of approximately 1¼ inches (30 mm).

According to one embodiment, while in use, the tubular member 100 may be disposed over the insertion tube 102. In order to assist the operator 320 gripping and manipulating the endoscope insertion tube 110, via the tubular member 100, the inside of the tubular member 100 may be provided with topological features, such as bumps or the like, on an inner surface (e.g., its bore) thereof.

The exterior non-slip surface 114 of the handle region 104 of the lubricant applicator device 150 may have a slot 704 (e.g., slit, slice) which may extend from one end of the tubular member 100 to the other end of the tubular member 100. The slot 704 may allow the tubular member 100 to be opened up slightly, preferably sufficiently that it may be removed substantially laterally (e.g., sideways) off of the endoscope insertion tube 110. Conversely, the tubular member 100 could be installed in a similar manner (spreading at the slot 704 and installing laterally) onto endoscope insertion tube 110, but a more appropriate manner of assembling the tubular member 100 to the endoscope insertion tube 110 may be to use the separate plastic tube (e.g., sleeve) 702, as may be described hereinabove.

The slot 704 may allow the tubular member 100 to be opened up (increased in diameter), slightly, the main purpose of which is to allow for the tubular member 100 to be removed easily from the endoscope insertion tube 110. The slot 704 may extend through the enlarged bulbous region 102 (e.g., gel chamber/gland 604) of the tubular member 100, in which case a chamber retaining wall 602 may be provided to contain the gel material 112.

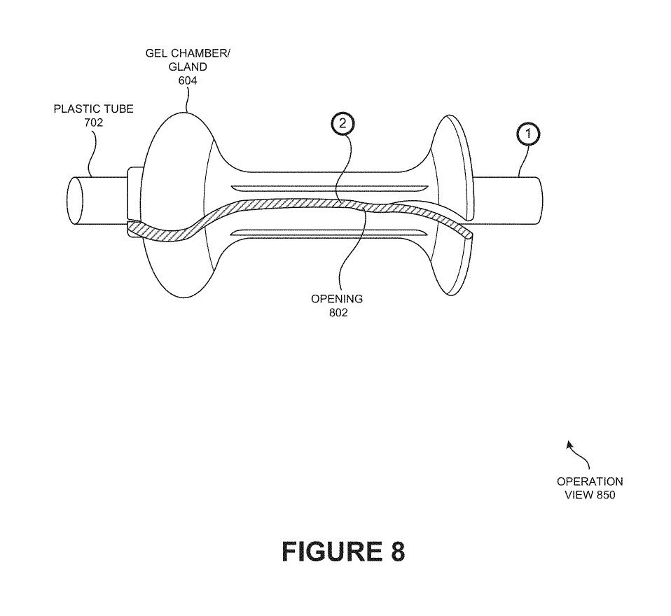

FIG. 8 is an operation view 850 of the lubricant applicator device of FIG. 1 illustrating the opening and closing of the lubricant applicator device 150 of FIG. 1, according to an embodiment. FIG. 8 is illustrates an elongated plastic tube 702 may be provided that fits within the tubular member 100 to: (1) seal gel material 112 in the gel chamber/gland 604; (2) act as a guide for inserting the endoscope insertion tube 110 into the tubular member 100. This may be beneficial when the distal end 106 of the endoscope insertion tube 110 is (i) flexible and/or (ii) has an irregular outer surface, which would otherwise make inserting the endoscope insertion tube 110 into the tubular member 100 difficult; and (3) act as a protective shield so that the endoscope insertion tube 110, particularly lenses at the distal end 106 of the endoscope insertion tube 110 are protected from gel material 112.

According to an embodiment, the plastic tube 702 may be inserted through the lubricant applicator device 150 and is removed once the endoscope 250 is inserted as depicted in the circle “1”. The lubricant applicator device 150 may be ‘sliced’ open 802 so it fits over the plastic tube 702 which is larger than the endoscope 250 diameter. Once the plastic tube 702 is removed, the lubricant applicator device 150 collapses to encapsulate the endoscope insertion tube 110 as depicted in the circle “2”. According to one embodiment, the lubricant applicator device 150 closes around the endoscope 250 once the plastic tube 702 is removed because it is molded in the size of the endoscope 250.

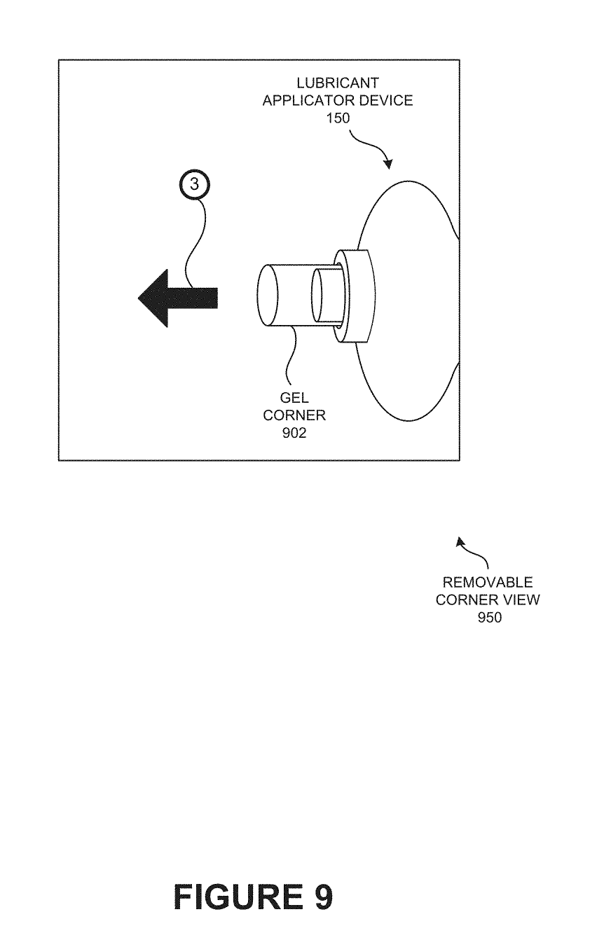

FIG. 9 is a removable corner view 950 of the lubricant applicator device 150 of FIG. 1 illustrating the removal of plastic tube 702 to expose the gel material 112 of the lubricant applicator device 150 at the gel corner 902 to the 150 of the endoscope 250, according to an embodiment.

According to another embodiment, the plastic tube 702 may be pulled out to expose the gel material 112 to the endoscope 250 while protecting the camera at the end of the endoscope 250 as depicted in the circle “3”. Prior to inserting the endoscope insertion tube 110 into the tubular member 100, the plastic tube 702 may be inserted into the tubular element 202, from either end thereof. The endoscope insertion tube 110 may then be guided through the tubular member 100 (or the tubular member 100 may be guided over the endoscope insertion tube 110) by the plastic tube 702. After inserting the endoscope insertion tube 110 through the tubular member 100 (or vice-versa), the plastic tube 702 may be removed, such as by extracting it from the front end of the tubular member 100. The plastic tube 702 may be in the form of a sheet of plastic material which is rolled up to form a tube, in which case, the plastic tube 702 could be extracted from the back end of the tubular member 100, unfurled, and discarded. Such a sheet would have a width greater than the circumference of the resulting endoscope insertion tube 110.

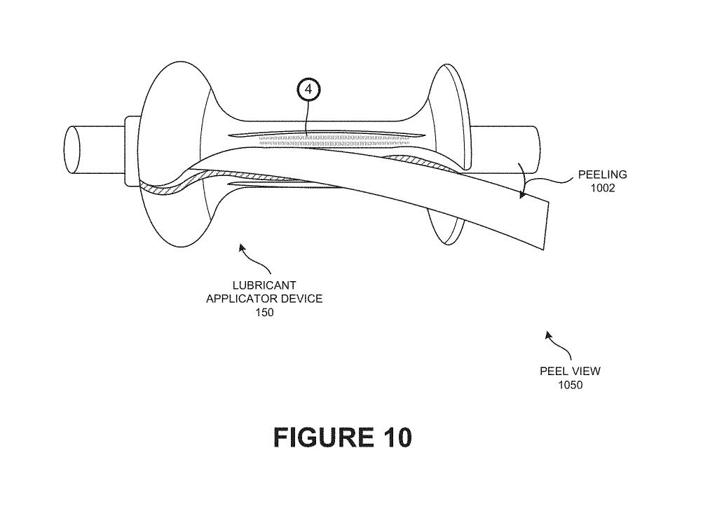

FIG. 10 is a peel view 1050 of the lubricant applicator device of FIG. 1 illustrating the removal and/or mounting of the lubricant applicator device 150, according to an embodiment.

According to one embodiment, the tubular element 100 may be disposed over the endoscope insertion tube 110. In use, the endoscope insertion tube 110 may be inserted, axially, through the tubular element 100, from the back end (e.g., proximal end 108) thereof, continuing through the tubular element 100, and emerging from the front end (e.g., distal end 106) of the tubular element 100. Alternatively (vice-versa), the tubular element 100 may be disposed, axially, over the endoscope insertion tube 110 by pushing the back end (e.g., proximal end 108) of the tubular element 100 over the front end (e.g., distal end 106) of the endoscope insertion tube 110. The back end (e.g., proximal end 108) of the tubular element 100 may be flared, to facilitate insertion of the endoscope insertion tube 110 into the tubular element 100 (or vice-versa). A plastic tube 702 described here in below, may assist in inserting the endoscope insertion tube 110 into the tubular element 100. Additionally, the tubular element 100 may be slit along its length to allow it to be installed upon and removed by peeling 1002 from the endoscope insertion tube 110 laterally, rather than axially as shown in circle “4”, according to one embodiment.

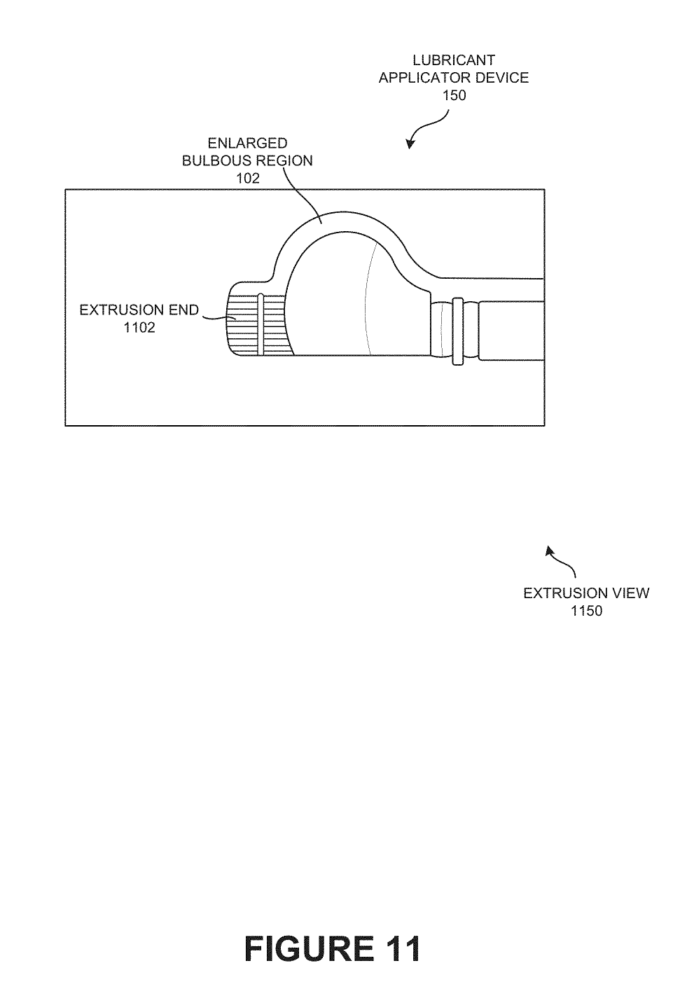

FIG. 11 is an extrusion view 1150 of the lubricant applicator device 150 of FIG. 1 illustrating the extrusion end of the lubricant applicator device 150, according to an embodiment.

Prior to inserting the endoscope insertion tube 110 into a patient, the operator 320 may lubricate the endoscope insertion tube 110 by squeezing the enlarged bulbous region 102 of the tubular member 100 causing gel material 112 to emerge, via the grooves (e.g., extrusion end 1102), onto the endoscope insertion tube 110. Subsequent to inserting the endoscope insertion tube 110 into a patient, the operator 320 may remove by peeling 1002 the tubular member 100 from the endoscope insertion tube 110 by pulling the tubular member 100 axially (endwise) off of the distal end 106 of the endoscope insertion tube 110. Alternatively, the tubular member 100 may be peeled off (e.g., by peeling 1002) of the endoscope insertion tube 110. The device can be removed off and/or mounted onto the endoscope 250 by peeling 1002, as shown in circle “4”, according to one embodiment.

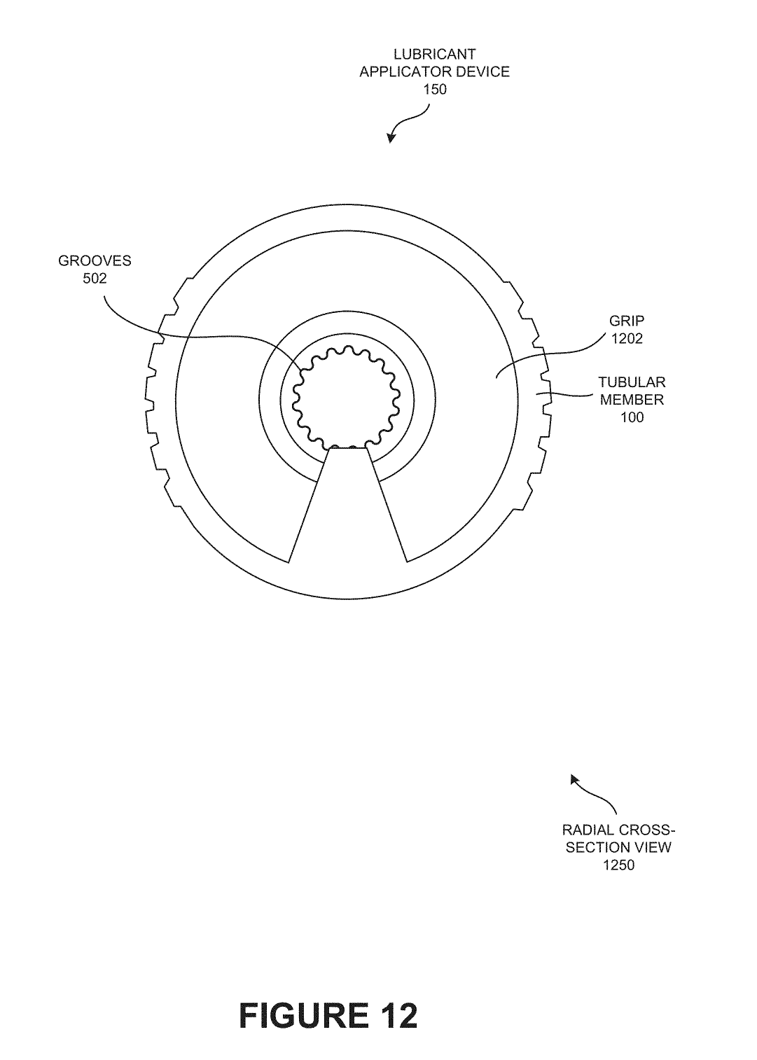

FIG. 12 is a radial cross section view 1250 of the enlarged bulbous region 102 of lubricant applicator device 150 of FIG. 1, according to one embodiment. At the distal end 106 of the enlarged bulbous region 102, the interior surface of the distal end 106 of the tubular member 100 may be provided with grooves 502 for delivering gel material 112 onto the endoscope insertion tube 110 when the enlarged bulbous region 102 is squeezed. The grooves 502 may be axial (as shown), and/or spiral. At the proximal end 108 of the enlarged bulbous region 102, the tubular member 100 may fit snugly onto the endoscope insertion tube 110, so that when the enlarged bulbous region 102 is squeezed, the gel material 112 within the enlarged bulbous region 102 exits the distal end 106 of the tubular member 100 onto the endoscope insertion tube 110, and a portion of the endoscope insertion tube 110 within the handle region 104 remain relatively free of gel material 112.

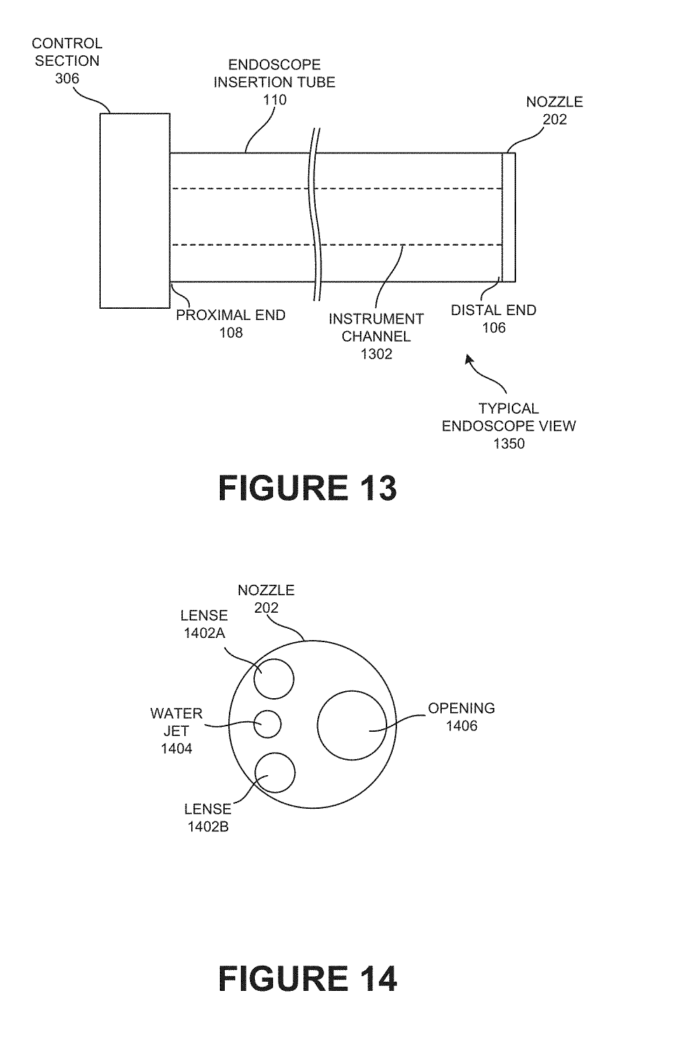

FIG. 13 is a typical view 1350 of an endoscope 250 of FIG. 2 illustrating the component parts of the endoscope, according to an embodiment.

The endoscope 250 illustrated in FIG. 13 may comprise a long flexible endoscope insertion tube 110 having two ends. The endoscope insertion tube 110 may have an outside diameter (OD) of approximately ⅝″ (15 mm). An instrument channel 1302 may extend through the extension tube from control section 306 disposed at the proximal end 108 of the endoscope insertion tube 110 to the distal end 106 of the endoscope insertion tube 110. The instrument channel 1302 may have a diameter of approximately 0.15″ (3.7 mm).

Various instruments and devices may extend through the endoscope insertion tube 110 from control section 306 disposed at the proximal end 108 of the endoscope insertion tube 110 to the distal end 106 of the endoscope insertion tube 110. The nozzle 202 may be disposed at the distal end 106 of the endoscope insertion tube 110.

FIG. 14 is an end view of the nozzle 202 of the endoscope 250 of FIG. 2 illustrating the component parts of the endoscope 250, according to an embodiment.

FIG. 14 illustrates the end view of the nozzle 202 of the nozzle 202 of the endoscope insertion tube 110 of FIG. 13. The nozzle 202 may be provided with lenses 1402A and 1402B for illuminating and viewing an area, a water jet 1404 for providing water to the area, and an opening 1406 at the end of the instrument channel 1302 which extends through the endoscope insertion tube 110. In another embodiment other features may be provided, such as an air/water nozzle.

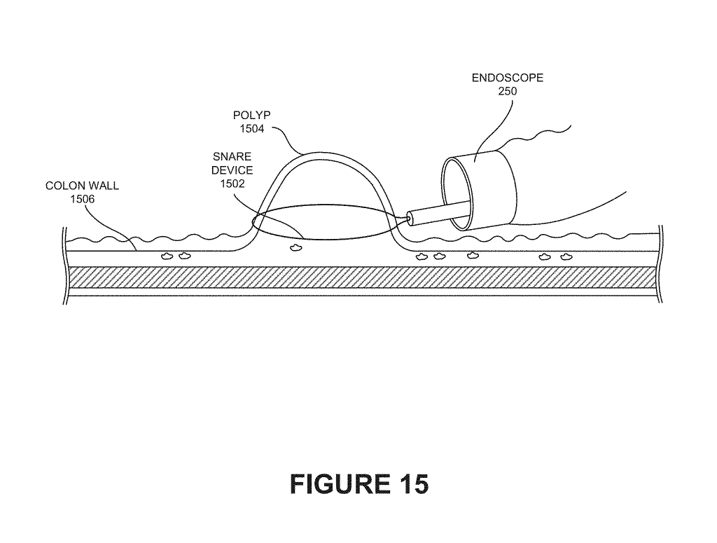

FIG. 15 illustrates a snare device 1502 connected to the endoscope of FIG. 2, according to an embodiment. The snare device 1502 and/or instruments (e.g., elongated biopsy device 1902) may be inserted through the instrument channel 1302 to perform procedures on an area of interest. For example, FIG. 15 shows the snare device 1502 which is a wire, encircling (e.g., lassoing) a polyp 1504. The wire may be heated to cauterize the polyp 1504.

The invention disclosed herein relates to an instrument and/or device that can be inserted by the operator 320 (e.g., doctor) through the endoscope insertion tube 110 of the endoscope 250, such as through the instrument channel 1302 of the endoscope insertion tube 110, and which emerges through the nozzle 202 of the endoscope insertion tube 110 to an area (such as the colon of a patient) being examiner/treated, and will be described in greater detail hereinbelow. The snare device 1502, and related techniques disclosed herein, may be particularly useful for detection of polyps 1504

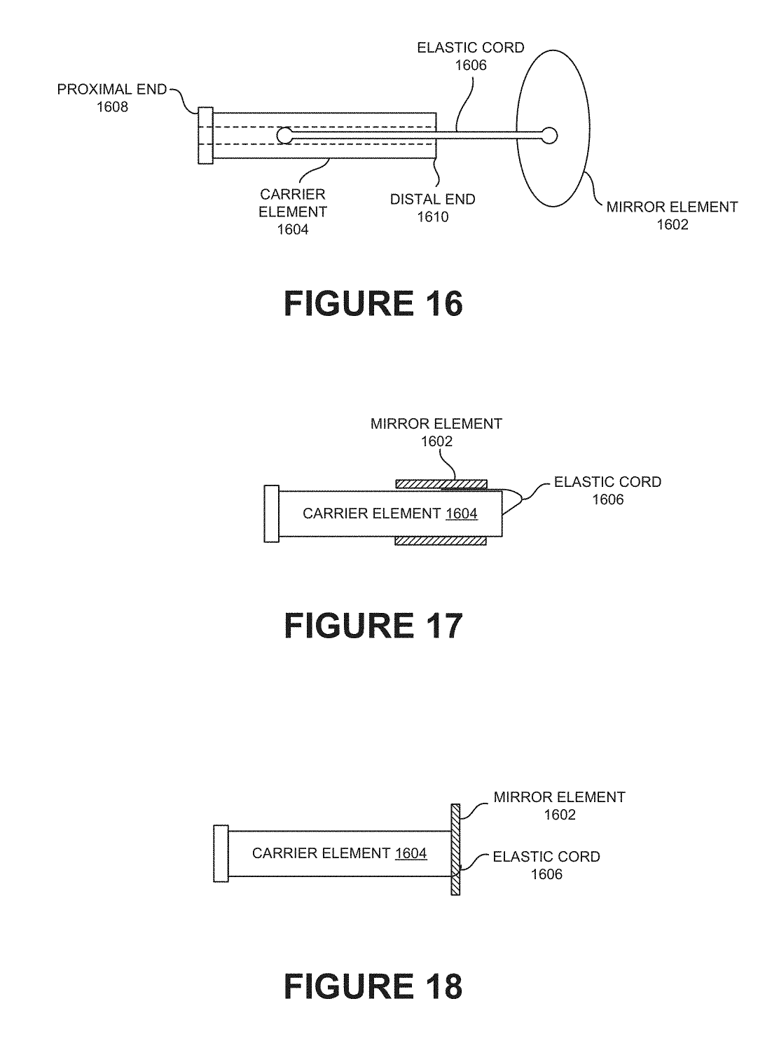

FIG. 16 illustrates a mirror element 1602 of the snare device 1502 connected to the endoscope 250 of FIG. 2, according to an embodiment. Particularly, FIG. 16 shows the snare device 1502 comprising a mirror element 1602, a carrier element 1604 and an elastic cord 1606.

The mirror element 1602 may be in the form of a disc, having a diameter of approximately 1″ (25.4 mm) and a thickness of approximately 0.001″ (0.025 mm.) The mirror element 1602 may be formed of reflective film, and may be initially flat (planar). The mirror element 1602 may be resilient so that it can spring back into its flat shape after being deformed. The carrier element 1604 may be elongate, such as in the form of a hollow cylinder (or tube) having a length of approximately 1.5″ (38 mm) and a diameter of approximately 1/16″ (2.5 mm).

The carrier element 1604 has a proximal end 1608 and a distal end 1610. The carrier element 1604 may be formed of rubber, silicon, or other compliant material. The carrier element 1604 could be a rod, rather than a cylinder.

The elastic (rubber) cord 1606 may extend from the distal end 1610 of the carrier element 1604 to the center of the mirror element 1602, and affixed thereto. The other end of the rubber cord 1606 may be secured within the carrier element 1604 (assuming that the carrier is a cylindrical tube). The rubber cord 1606 may be stretched while wrapping the mirror element 1602 around the carrier.

A procedure is described wherein the mirror element 1602 is reduced in size, deployed to an area of interest, and is then restored to its original flat configuration for improving viewing of the area of interest. In a first step of a procedure for examining the area, the flexible mirror element 1602 is wrapped around the carrier element 1604 (“carrier”) and secured thereto with a water-soluble adhesive and/or casing so that it does not unwind (unfurl).

FIG. 17 is a cross-sectional view of the mirror element 1602 of the snare device 1502 of FIG. 16, according to an embodiment. The mirror element 1602 is shown in cross-section, wrapped around the carrier element 1604. The diameter of the snare device 1502 (carrier element 1604 with mirror element 1602 wrapped around it) is only slightly larger than the diameter of the carrier element 1604. The diameter of the snare device 1502 may be approximately 3/32″ (3.5 mm) (sufficiently less than the diameter of the instrument channel 1302 that it may pass therethrough).

Next, the snare device 1502 (carrier with mirror element 1602 wrapped around it) may be mounted to the end of endoscope 250, secured thereto by the wire lasso. Then, the snare device 1502 may be inserted through the instrument channel 1302 of the endoscope 250 to the area of interest. FIGS. 13 and 14 illustrates instrument channel 1302 and opening 1406 in distal end 106 of the endoscope 250.

Next, the mirror element 1602 may be deployed by splashing the snare device 1502 with water, which will dissolve the adhesive, allowing the mirror element 1602 to unwrap (open, unfurl) itself.

FIG. 18 illustrates the contraction view of the snare device of FIG. 16, according to an embodiment.

According to one embodiment, the elastic cord 1606, which is under tension, will contract to pull the mirror flush with the flat end of the carrier element 1604 to flatten the mirror element 1602.

Once deployed, the mirror element 1602 may provide visibility to the back sides of polyps 1504 and tissue. The flexibility of the mirror element 1602 provides much access and adjustment to the reflection angles into the camera and to optimize light illumination. The mirror element 1602 may be bent by manipulation of a snare device 1502 and/or via endoscope 250 positioning so it is in contact with tissue. The flexibility of the mirror element 1602 permits the mirror element 1602 to bend behind tissue creating a curved mirror surface that enhances visibility into difficult crevasses.

When the mirror element 1602 is bent to form a convex shape, the image formed by a convex mirror is smaller than an image in a plane (flat) mirror. Because the image is smaller, more images can fit onto the mirror, so a convex mirror provides for a larger field of view than a plane mirror. This is useful to investigate a large area that may not be easily accessible.

When the mirror element 1602 is bent to form a concave shape, it may provide “magnification” of the visible area which is useful for closer investigation of specific areas.

The flexibility of the mirror element 1602 ensures that there is no damage to surrounding tissue.

Once the procedure is complete, the snare device 1502 may be pulled out through the instrument channel 1302 and/or released into the body, to be disposed of naturally. The snare device 1502 and instrument channel 1502 is then free to be used to remove polyps 1504 in the traditional way.

According to one embodiment, a method of improving viewing an area of interest with an endoscope 250 may include providing a flat, resilient mirror element 1602, providing a carrier element 1604, providing an elastic cord 1606 extending from an end of the carrier element 1604 to the center of the mirror element 1602, and wrapping the mirror element 1602 around the carrier element 1604. Further, the method may include securing the mirror element 1602 thereto with a water soluble adhesive and/or casing, inserting the carrier element 1604 with mirror element 1602 wrapped around it through the instrument channel 1302 of the endoscope 250 to the area of interest, splashing water on the mirror element 1602 to dissolve the water-soluble adhesive and allowing the resilient mirror element 1602 to unfurl and deploy, and positioning the deployed mirror element 1602 as desired to improve viewing.

According to another embodiment, the snare device 1502 for improving viewing an area of interest with an endoscope 250 may include a flat, flexible mirror element 1602, an elongate carrier element 1604, and an elastic cord 1606 extending from an end of the carrier element 1604 to the mirror element 1602. The mirror element 1602 may include a reflective film.

The mirror element 1602 may be resilient so that it can spring back into its flat shape after being deformed. The snare device 1502 may include the carrier element 1604 such as rubber, silicon, or other compliant material. In another embodiment, a method of viewing an area of interest with an endoscope 250 may include providing a mirror element 1602 and inserting the mirror element 1602 through the endoscope insertion tube 110 of an endoscope 250 to be near the area of interest. The mirror element 1602 may be furled for passing through the endoscope insertion tube 110 and unfurled at the area of interest. The method may include bending the mirror element 1602 to have a convex or concave shape.

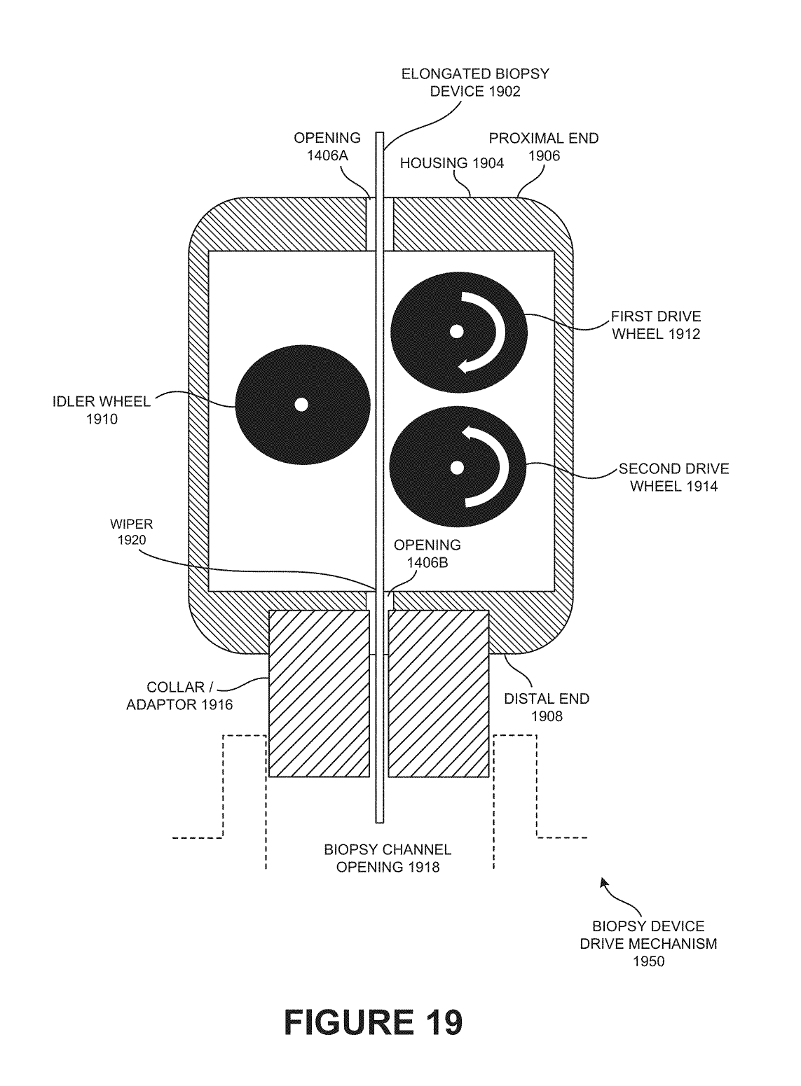

FIG. 19 illustrates a biopsy device mechanism 1950 connected to the endoscope 250 of FIG. 2, according to an embodiment. The figure shows the elongated biopsy device 1902, a housing 1904, a proximal end 1906, a distal end 1908, an idler wheel 1910, a first drive wheel 1912, a second drive wheel 1914, a collar/adapter 1916, a biopsy channel opening 1918, a wiper 1920.

The elongated biopsy device 1902 is shown entering through the opening 1406A at the proximal end 1906 of the housing 1904, past the idler wheel 1910 and drive wheels (e.g., first drive wheel 1912, second drive wheel 1914) within the housing 1904, and exiting the housing 1904 through the opening 1406B in the distal end 1908 of the housing 1904. The elongated biopsy device 1902 may be driven in a distal 1908 (into the endoscope 250) and/or proximal 1906 (out of the endoscope 250) direction by being captured between the idler wheel 1910 and a selected one of the drive wheels (e.g., first drive wheel 1912, second drive wheel 1914).

The housing 1904 may have the proximal end 1906 and the opposite distal end 1908. The housing 1904 may include the idler wheel 1910 and drive wheels (e.g., first drive wheel 1912, second drive wheel 1914). The proximal end 1906 and/or the distal end 1908 of the housing 1904 may have opening (e.g., 1406A and 1406B) to allow insertion and extraction of the elongated biopsy device 1902. The idler wheel 1910 may be disposed within the housing 1904. The distal end 1908 of the housing 1904 may fit directly over the biopsy channel opening 1918, without the need for an adapter.

The idler wheel 1910 may be disposed closely adjacent the two drive wheels (e.g., first drive wheel 1912, second drive wheel 1914). The first drive wheel 1912 may be disposed within the housing 1904 to rotate in a clockwise direction. The second drive wheel 1914 may be disposed within the housing 1904 to rotate in a counter-clockwise direction. The collar/adaptor 1916 may be provided at the distal end 1908 of the housing 1904 for releasably mounting the biopsy device drive mechanism 1950 to the biopsy channel opening 1918 of a control section 306 of the endoscope 250. The wiper 1920 may be provided at the distal end 1908 opening 1406B of the housing 1904 to remove moisture off of the elongated biopsy device 1902 when it is withdrawn (in the proximal direction) from the insertion tube 110 of the endoscope 250.

The biopsy device mechanism 1950 may allow the insertion and/or retraction of the elongated biopsy device 1902 into and/or out of the insertion tube 110 of the endoscope 250. The biopsy device mechanism 1950 may include a clutch mechanism that disengages the biopsy device mechanism 1950 if the elongated biopsy device 1902 encounters substantial resistance to motion to prevent the damage to the elongated biopsy device 1902, endoscope 250, or accidental perforation of tissue. The biopsy device mechanism 1950 may include an encoder associated with the idler wheel 1910 to track the position of the elongated biopsy device 1902 such that when the elongated biopsy device 1902 is tracked, the elongated biopsy device 1902 movement and position can be automated via a motion controller.

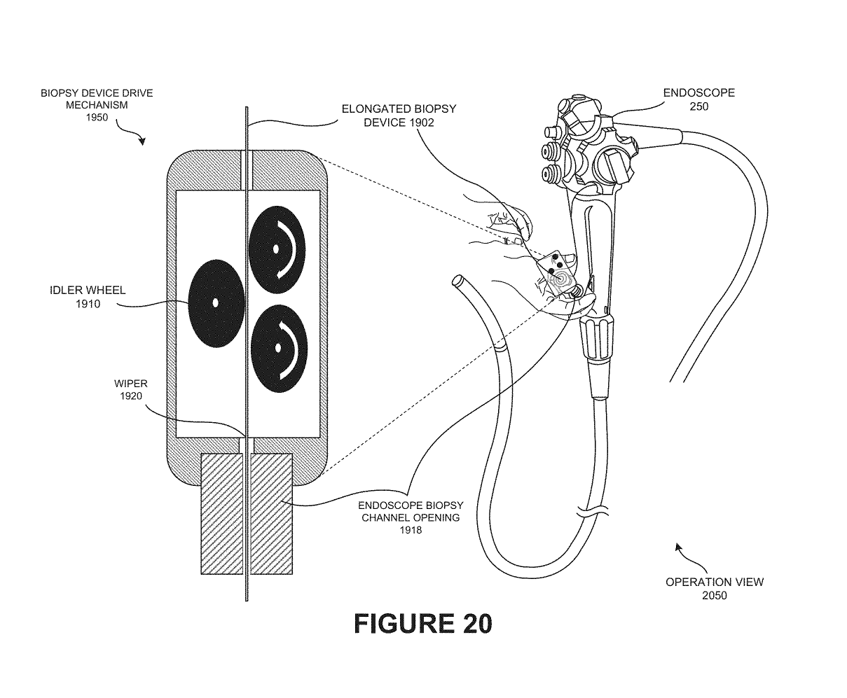

FIG. 20 illustrates an operation view 2050 of an elongated biopsy device 1902 connected to the endoscope 250 of FIG. 2, according to an embodiment. In one embodiment, the idler wheel 1910 may be manipulated (e.g., by the operator 320) to engage the biopsy device between the idler wheel 1910 and a selected one of the oppositely-rotating drive wheels (e.g., a first drive wheel 1912 or a second drive wheel 1914), to selectively engage the elongated biopsy device 1902 between the idler wheel 1910 and a selected one of the drive wheels (e.g., a first drive wheel 1912 or a second drive wheel 1914), for selectively inserting and withdrawing the elongated biopsy device 1902 from the endoscope insertion tube.

The friction may move the elongated biopsy device 1902 linearly, into and out of the biopsy channel opening 1918 when the elongated biopsy device 1902 is disposed between the rotating drive wheel (e.g., first drive wheel 1912, second drive wheel 1914) and the idler wheel 1910. The elongated biopsy device 1902 may be inserted and/or extracted depending on the direction of the wheel rotation (or which wheel is engaging the elongated biopsy device 1902).

The biopsy device drive mechanism 1950 may be provided with a wiper 1920 to remove moisture from the tubular body of the elongated biopsy device 1902 to enhance gripping. The operator 320 may push (e.g., operate) a lever to engage the driving wheel (e.g., first drive wheel 1912, second drive wheel 1914) to the elongated biopsy device 1902 and/or push (e.g., operate) a switch that activates the driving motor. This friction contact drives the elongated biopsy device 1902 in and out of the endoscope 250. To collect manual samples from the area of interest, the operator 320 may rely on the endoscope optics to properly position the distal end 1908 (with jaws) of the elongated biopsy device 1902.

In another embodiment, the housing of the elongated biopsy device 1902 may have two drive wheels. The drive wheels may be made to rotate in a particular direction. The air motor may be arranged so that a first drive wheel 1912 rotates in a first direction (such as clockwise) and a second drive wheel 1914 rotates in an opposite (such as counterclockwise) direction. The operator 320 may selectively couple the air motor to one of the oppositely-rotating drive wheels to control the direction (e.g., insertion and/or extraction) of the elongated biopsy device 1902.

Yet in another embodiment, a biopsy device mechanism 1950 may have a drive wheel (e.g., first drive wheel 1912 or second drive wheel 1914). The drive wheel (e.g., first drive wheel 1912 or second drive wheel 1914) may be capable of rotating in two different directions (e.g., clockwise and clockwise). An idler wheel 1910 of the biopsy device mechanism 1950 may be spaced from the drive wheel (e.g., first drive wheel 1912 or second drive wheel 1914). The elongated biopsy device 1902 may fit between and be linearly advanced into and/or withdrawn from the biopsy channel opening 1918 when the drive wheel (e.g., first drive wheel 1912 or second drive wheel 1914) is being driven. While operating with one drive wheel (e.g., first drive wheel 1912 or second drive wheel 1914), the air motor may be made to change direction via a finger actuated switch and/or foot pedal. The biopsy device mechanism 1950 may operate the drive wheel (e.g., first drive wheel 1912 or second drive wheel 1914). The drive wheel (e.g., first drive wheel 1912 or second drive wheel 1914) may include the air motor which receives air from the endoscope 250 air source. To be disposable, the entire biopsy device drive mechanism 1950 assembly may be made from plastic. The air motor may be formed inexpensively from plastic, and discarded (or recycled) after use.

According to an aspect of this invention, generally, a mirror element (e.g., which may be referred to as a “filament”) of a snare device may be deployed via an endoscope to an area of interest being examined to facilitate viewing otherwise inaccessible portions of the area being examined. The mirror element may in the form of a flat, flexible disc made of a resilient material. The mirror element may be wrapped around and temporarily secured to a carrier element with a water-soluble adhesive. The device (mirror and carrier) may be inserted through the endoscope, then water may be applied to allow the mirror to unfurl (open itself up), and the mirror may be positioned (manipulated) to better view the area of interest with the endoscope.

Some benefits of the snare device and procedure disclosed herein may include:
Increased mucosa! inspection of the right colon
Significant cost reduction
Single Use
Versatile (can be used to visualize both the lower GI tract and the Upper GI tract)
Facilitates Cecal visualization (for patients with redundant colons)
Increases polyp detection resulting in Colon Cancer reduction
Reduction of mortality and morbidity from Colon Cancer
Safety:
   Decreases rate of perforation during difficult colonoscopies and when using retroflexion technique in the Cecum and Rectum
   Allows visualization without reaching the Cecum, therefore reducing complications rate
   Enables reduction of anesthesia time.

In yet another aspect of this invention, disclosure herein relates to an instrument and/or device that can be inserted by the operator (e.g., doctor) through the endoscope insertion tube of the endoscope, such as through the instrument channel of the endoscope insertion tube, and which emerges through the nozzle of the endoscope insertion tube to an area (e.g., the colon of a patient) being examiner/treated, and will be described in greater detail herein below. The device, and related techniques disclosed herein, may be particularly useful for performing biopsies, but in addition to biopsy forceps and snare devices, this invention also may be used to introduce other endoscopy instruments, such as hot tips, needles, and other devices through the instrument channel, to be introduced into the body cavity through the endoscope.

While the invention(s) has/have been described with respect to a limited number of embodiments, these should not be construed as limitations on the scope of the invention(s), but rather as examples of some of the embodiments. Those skilled in the art may envision other possible variations, modifications, and implementations that are also within the scope of the invention(s), and claims, based on the disclosure(s) set forth herein, according to one embodiment.

What is claimed is:

1. A video cart system, comprising:
a video system center having a processor and a memory;
a monitor coupled with the video system center;
a light source coupled with the video system center;
an endoscope coupled the video system center having a control section;
an insertion tube having a camera at a distal end of the insertion tube that permits viewing of an area of an internal structure that is directly in front of the camera; and
a mirror to be mechanically extendable at a distance from the camera and reflected backward, so that the camera permits viewing of an area behind the camera through a reflection of an image produced by the mirror when an operator of the endoscope utilizes the control section of the endoscope.

2. A lubricant applicator device, comprising:
a tubular member to surround a portion of an endoscope insertion tube having a mirror that can be mechanically extended a distance from the camera and reflected backward, so that the camera can view an area behind the camera through a reflection of an image produced by the mirror when an operator of the endoscope utilizes a control section of an endoscope such that the tubular member is extendable over the portion of the endoscope insertion tube to snugly surround the portion of the endoscope insertion tube;
an enlarged bulbous region adjacent to a distal end of the tubular member to be pre-filled with a gel material that is releasable onto a surface area of the endoscope insertion tube encompassed by the enlarged bulbous region; and
a handle region adjacent to a proximal end of the tubular member that extends from the enlarged bulbous region to provide a non-slip exterior surface to enable a human hand of an operator to grasp the handle region and manipulate the endoscope insertion tube while rotatably affixing a thumb of the hand onto the enlarged bulbous region to release the gel material when a pressure is applied to the enlarged bulbous region using the thumb.

3. The lubricant applicator device of claim 2 wherein the lubricant applicator device is any one of a single use lubricant applicator device and a multiple use lubricant applicator device.

4. The lubricant applicator device of claim 2 wherein a flow rate of the gel material onto the surface area of the endoscope insertion tube encompassed by the enlarged bulbous region is proportional to the pressure applied to the enlarged bulbous region by the thumb of the human hand of the operator,
wherein the gel material is refillable in the enlarged bulbous region when the lubricant applicator device is the multiple use lubricant applicator device, and
wherein the gel material is pre-filled in the enlarged bulbous region at a factory when the lubricant applicator device is the single use lubricant applicator device.

5. The lubricant applicator device of claim 2 wherein the tubular member is a single mold assembly comprising of both the enlarged bulbous region and the handle region and is formed of a material comprising at least one of a rubber, a polymeric flexible material, a nylon material, a biodegradable material, a silicon based material, and a semi-rigid material.

6. The lubricant applicator device of claim 2 wherein the endoscope insertion tube is inserted into the lubricant applicator device axially, through the proximal end of the tubular member and continuing through the tubular member to emerge from the distal end of the tubular member adjacent to the enlarged bulbous region.

7. The lubricant applicator device of claim 2 wherein a section of the endoscope insertion tube encapsulated by the handle region to remain segregated from the gel material of the enlarged bulbous region such that an exterior surface of the handle region to be unaffected by an extraction of the gel material from the enlarged bulbous region when the pressure of the thumb of the operator is applied on the enlarged bulbous region causing the gel material to release onto the surface area of the endoscope insertion tube encompassed by the enlarged bulbous region of the lubricant applicator device,
wherein the enlarged bulbous region is a chamber that comprises the gel material.

8. The lubricant applicator device of claim 2 wherein the endoscope insertion tube is transgressible through the lubricant applicator device when it is pushed through the tubular member from the proximal end of the tubular member.

9. The lubricant applicator device of claim 2 wherein the gel material is a water-soluble gel that is gentle on mucosal surfaces of a patient.

10. A method of forming a lubricant applicator device, comprising:
generating a single mold having a tubular member with an enlarged bulbous region at a distal end of the lubricant applicator device and a handle region at a proximal end of the lubricant applicator device,
wherein the tubular member to surround a portion of an endoscope insertion tube such that the tubular member is placeable over the portion of the endoscope insertion tube to surround the portion of the endoscope insertion tube, and
wherein the handle region to provide a non-slip exterior surface to enable a human hand of an operator to grasp the handle region and manipulate the endoscope insertion tube while rotatably affixing a thumb of the hand onto the enlarged bulbous region to release the gel material when a pressure is applied to the enlarged bulbous region using the thumb,
wherein the endoscope insertion tube includes a mirror that can be mechanically extended a distance from the camera and reflected backward, so that the camera can view an area behind the camera through a reflection of an image produced by the mirror when an operator of the endoscope utilizes the control section of the endoscope.

11. The method of claim 10 wherein the lubricant applicator device has a sideways water-tower like shape.

12. The method of claim 10 wherein the lubricant applicator device is formed of a material comprising at least one of a rubber, a polymeric flexible material, a nylon material, a biodegradable material, a silicon based material, and a semi-rigid material.

13. The method of claim 10 wherein the lubricant applicator device is any one of a single use lubricant applicator device and a multiple use lubricant applicator device,
wherein the gel material is refillable in the enlarged bulbous region when the lubricant applicator device is the multiple use lubricant applicator device, and
wherein the gel material is pre-filled in the enlarged bulbous region at a factory when the lubricant applicator device is the single use lubricant applicator device.
